(19) United States
(12) Patent Application Publication
(10) Pub. No.: US 2022/0162145 A1
(43) Pub. Date: May 26, 2022

(54) PROCESS FOR SEPARATION OF SATURATED AND UNSATURATED CARBOXYLIC ACIDS

(71) Applicant: SHELL INTERNATIONALE RESEARCH MAATSCHAPPIJ B.V., 2596 HR THE HAGUE (NL)

(72) Inventors: Kai Jürgen FISCHER, Amsterdam (NL); Jean-Paul Andre Marie Joseph LANGE, Amsterdam (NL)

(21) Appl. No.: 17/602,881

(22) PCT Filed: May 4, 2020
(86) PCT No.: PCT/EP2020/062291

§ 371 Date: Oct. 11, 2021

Related U.S. Application Data
(60) Provisional application No. 62/843,647, filed on May 6, 2019.

(51) Int. Cl. C07C 51/44; B01D 3/40; C07C 51/48

(52) U.S. Cl. CPC C07C 51/44; C07C 51/48; B01D 3/40

(57) ABSTRACT

The invention provides a process for separating saturated and unsaturated carboxylic acids is described. The process includes providing a stream comprising same carbon number saturated and unsaturated carboxylic acids; contacting said stream with an extractive solvent in an extractive distillation unit, to produce a first stream comprising extractive solvent and unsaturated carboxylic acids and a second stream comprising saturated carboxylic acids, and feeding said first stream to a solvent recovery unit, to produce a third stream comprising unsaturated carboxylic acids and a fourth stream comprising extractive solvent. In some embodiments, the extractive solvent has a boiling point at atmospheric pressure that is at least 5° C. higher than the boiling point of the unsaturated carboxylic acid.

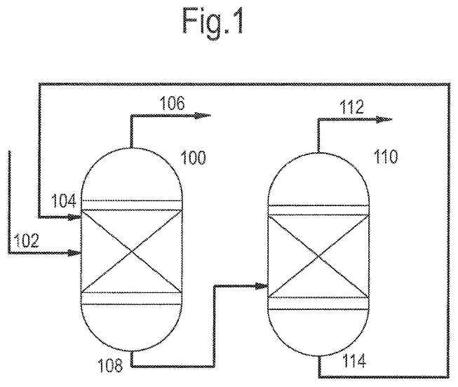

### CROSS-REFERENCE TO RELATED APPLICATION

The application claims the benefit of the U.S. Provisional Patent Application 62/843,647 filed May 6, 2019 entitled PROCESS FOR SEPARATION OF SATURATED AND UNSATURATED CARBOXYLIC ACIDS, the entirety of which is incorporated by reference herein.

### FIELD OF THE INVENTION

The present invention relates to a process for the separation of saturated and unsaturated carboxylic acids by means of extractive distillation.

### BACKGROUND OF THE INVENTION

Several oxidative chemical conversion processes known in the art produce aqueous streams comprising saturated and unsaturated carboxylic acids as side products. For example, it is known to oxidatively dehydrogenate alkanes having 3 to 6 carbon atoms (“C3-C6 alkanes”) such as propane, butane or isobutane resulting in propylene, butene or isobutene, respectively, in an oxidative dehydrogenation (oxydehydrogenation; ODH) process. The dehydrogenated equivalent of the alkane may be further oxidized under the same conditions into the corresponding saturated or unsaturated carboxylic acid, such as acetic acid, acrylic acid, propionic acid, or methacrylic acid. Other examples include the dehydrogenation of alcohols, the oxidation of aldehydes and the conversion (fermentation, pyrolysis, liquefaction) of biomass. Furthermore, biomass conversion processes produce C3-oxygenates which may be further converted to acrylic acid, along with the saturated or unsaturated carboxylic acids referenced above.

In the above processes as well as in other oxidative conversion process, the saturated and unsaturated carboxylic acids thus produced are generally considered as waste products. Although they could be condensed together with water from the reactor effluent as an aqueous carboxylic acid (ca. 10 wt %) stream, the low relative volatility of saturated versus unsaturated carboxylic acids renders ordinary distillation separation of saturated and unsaturated carboxylic acid troublesome, as this would require very large condensate recycle and/or separation trains.

However, saturated and unsaturated C3-C6 carboxylic acids are valuable ingredients and building blocks for use in the chemical industry. For example, the global demand for acrylic acid is around 5 million tonnes per year (Mt/a), with applications as superabsorbent in e.g. incontinence and personal care products, applications in surface coatings, adhesives and sealants, in textiles, in the water treatment industry, in mineral processing and numerous other applications in the form of acrylate esters.

It is an objective of the present invention to provide a technically advantageous, efficient and affordable process for separating saturated C3-C6 carboxylic acids from unsaturated C3-C6 carboxylic acids, such as propionic acid from acrylic acid, from vaporous and/or liquid aqueous streams.

### SUMMARY OF THE INVENTION

In some embodiments, a process for separating saturated and unsaturated carboxylic acids is described. The process includes providing a stream comprising same carbon number saturated and unsaturated carboxylic acids; contacting said stream with an extractive solvent in an extractive distillation unit, to produce a first stream comprising extractive solvent and unsaturated carboxylic acids and a second stream comprising saturated carboxylic acids, and feeding said first stream to a solvent recovery unit, to produce a third stream comprising unsaturated carboxylic acids and a fourth stream comprising extractive solvent. In some embodiments, the extractive solvent has a boiling point at atmospheric pressure that is at least 5° C. higher than the boiling point of the unsaturated carboxylic acid.

### BRIEF DESCRIPTION OF THE DRAWINGS

The FIGURE shows an embodiment of the present invention, wherein a stream comprising same carbon number saturated carboxylic acids and unsaturated carboxylic acids is contacted with an extractive solvent in an extractive distillation unit.

### DETAILED DESCRIPTION OF THE INVENTION

While the process of the present invention and the streams used in said process are described in terms of “comprising”, “containing” or “including” one or more various described steps and components, respectively, they can also “consist essentially of” or “consist of” said one or more various described steps and components, respectively.

In the process of the present invention, the term “aqueous stream” may refer both to a water-containing stream in the liquid phase and to a water-containing stream in the vapour phase, said aqueous stream further comprising same carbon number saturated and same carbon number unsaturated carboxylic acids in the liquid or vapour/gas phase, respectively. The aqueous stream comprising same carbon number saturated and unsaturated carboxylic acids may be any stream comprising at least 0.1, or at least 1 wt %, more preferably at least 3 wt %, even more preferably at least 5 wt %, yet even more preferably at least 10 wt % or 15 wt %, most preferably at least 20 wt % of same carbon number saturated carboxylic acids and unsaturated carboxylic acids. The aqueous stream may include at least 0.1 wt %, or at least 1 wt %, more preferably at least 3 wt %, even more preferably at least 5 wt %, yet even more preferably at least 10 wt % or 15 wt %, most preferably at least 20 wt % wt % of saturated carboxylic acids. In some embodiments, the aqueous stream may include at least 0.1, or at least 1 wt %, more preferably at least 3 wt %, even more preferably at least 5 wt %, yet even more preferably at least 10 wt % or 15 wt %, most preferably at least 20 wt % of unsaturated carboxylic acids. In some embodiments, the aqueous stream may also include water and contaminants, such as lighter acids. Typically, said aqueous stream comprising same carbon number saturated and unsaturated carboxylic acids originates from an oxidative chemical conversion process of C3-C6 alkanes and/or alkenes, wherein the same carbon number saturated and unsaturated carboxylic acids is obtained as a side product. In other embodiments, the stream comprising same carbon number saturated and unsaturated carboxylic acids originates from an oxidative chemical conversion process of C3-C6 alkanes and/or alkenes obtained as a side product. It is preferred that the aqueous feed stream of the extractive distillation process comprises same carbon number saturated and unsaturated carboxylic acids in a concentration of at least 1 wt %, more preferably at least 3 wt %, even more preferably at least 5 wt %, yet even more preferably at least 10 wt %, most preferably at least 20 wt %.

In some embodiments, an additional concentration step, for example of a dilute liquid or gaseous process effluent comprising same carbon number saturated and unsaturated carboxylic acids, may be applied prior to contacting the same carbon number saturated and unsaturated carboxylic acids with the extractive solvent in the extractive distillation unit. Such concentration step may comprise any suitable method for removing excess water from an aqueous same carbon number saturated and unsaturated carboxylic acids stream, including reverse osmosis, liquid-liquid extraction, adsorption or water pervaporation. In some embodiments, extractive distillation may be used to recover acid from water, such as that disclosed in WO2017114816, which is hereby incorporated in its entirety herewith.

In some embodiments of the invention, a dilute liquid aqueous stream comprising same carbon number saturated and unsaturated carboxylic acids is subjected to liquid-liquid extraction (LLE) using an extractive solvent to obtain a more concentrated stream comprising same carbon number saturated and unsaturated carboxylic acids and water, which is subsequently used as the feed stream of an extractive distillation process as described herein in order to remove entrained water. In some embodiments, the extractive solvent to obtain a more concentrated stream may be the same as a subsequent extractive solvent or may be a water soluble extractive solvent. In another embodiment of the invention, a gaseous or vaporous effluent comprising same carbon number saturated and unsaturated carboxylic acids is treated using water pervaporation to withdraw most of the water from the saturated and unsaturated carboxylic acids stream, which is subsequently separated using extractive distillation as described herein. In yet another embodiment, a vaporous effluent comprising same carbon number saturated and unsaturated carboxylic acids is concentrated by adsorption onto a solid, followed by desorption of a more concentrated saturated carboxylic acid/water vapour stream subsequently separated using extractive distillation as described herein.

Typically, such a concentration step yields an aqueous feed stream comprising same carbon number saturated and unsaturated carboxylic acids in a total concentration of at least 5 wt %, more preferably at least 10 wt %, even more preferably at least 15 wt %, most preferably at least 20 wt %, at least 50 wt %, at least 80 wt %, or at least 90 wt %.

In some embodiments, the aqueous stream comprising same carbon number saturated and unsaturated carboxylic acids is in the vapour phase. Such a vaporous phase stream comprising water and same carbon number saturated and unsaturated carboxylic acids may be the effluent stream from a gas-phase (oxidative) conversion process of alkanes and/or alkenes. By directly subjecting the vaporous effluent comprising same carbon number saturated and unsaturated carboxylic acids and water of such process to the extractive distillation step, capital and operating expenditure on excessive condensation and reheating steps can be avoided.

In one embodiment, the aqueous stream comprising same carbon number saturated and unsaturated carboxylic acids originates from the oxidation of propane. The oxidation process typically produces a product stream comprising propene, acrylic acid, some propionic acid as well as water and carbon dioxide. In another embodiment, the aqueous stream comprising same carbon number saturated and unsaturated carboxylic acids originates from the oxidation of propene. In the extractive distillation process of the invention, the gaseous or liquid aqueous stream comprising same carbon number saturated and unsaturated carboxylic acids is contacted with an extractive solvent in a suitable extractive distillation unit in order to separate the same carbon number saturated carboxylic acid from the unsaturated carboxylic acid.

In another embodiment, the aqueous stream comprising same carbon number saturated and unsaturated carboxylic acids originates from the dehydration of lactic acid to acrylic acid.

Extractive distillation is a distillation process wherein an extractive solvent is added in order to modify the relative volatility of the components that need to be separated, thus enabling a larger degree of separation or requiring less effort to affect the same separation. The extractive solvent is typically a high-boiling, relatively non-volatile compound. The extractive solvent typically boils at a higher temperature than any of the close-boiling components being separated and has particular affinity with one of the two close-boiling components. In this way the component of the resulting mixture that has the least affinity with the solvent is obtained at the top of the extractive distillation column and the other component along with the extractive solvent is obtained from the bottom section. Owing to the high boiling point of the extractive solvent, this bottom stream can then be separated in a secondary distillation (or rectification) column in order to provide a purified product and recover the extractive solvent. Extractive distillation should be distinguished from the best-known form of azeotropic distillation, i.e. wherein the solvent (or entrainer) forms a low-boiling azeotrope with the compound to be separated and is thus vaporized into the top rather than collected at the bottom of the distillation column. In some embodiments, the extractive solvent interacts with the unsaturated acid, resulting in the lowering of the vapor pressure.

In the present invention, any suitable extractive distillation unit available in the art may be employed. Typically, such extractive distillation unit comprises a tray (plate) column having an inlet for receiving a feed stream comprising the component to be separated (such as acrylic and propionic acid), wherein the extractive solvent is fed to a tray above this feed stream. The extractive solvent preferentially associates with the component to be separated, taking it down the column where it is obtained as a bottom stream, whereas the lower-boiling water component of the resulting mixture is obtained as the top distillate stream.

Generally, choice of extractive solvent is of importance in the extractive distillation process, since suitable extractive solvents can decrease the solvent ratio and/or the liquid load of the extractive distillation unit, thus rendering an easy and more economical implementation of the extractive distillation column in a process line-up.

The extractant employed in the process of the invention is suitable for interacting with the unsaturated carboxylic acids to raise its boiling point. It is generally a solvent system which selectively interacts with the one or more unsaturated carboxylic acid. The solvent system may comprise a single solvent or a plurality of solvents. The extractant is also referred to herein as the “extraction solvent.” The terms “extractant” and “extraction solvent”, as used herein, shall be taken to have an identical meaning and to be interchangeable.

Without wishing to be bound by theory, it is believed that the extractive solvent(s) interacts with the C═C bond, such that it is attracted and the volatility is decreased by lowering the activity coefficient.

In some embodiments, the saturated and unsaturated carboxylic acids have nearly identical boiling points and similar polarity. However, the saturated and unsaturated carboxylic acids may offer different affinity to the extractive solvent by having differences in acidity and Hansen parameters. These differences, either independently or combined, can be exploited for separation means by extractive distillation. In some embodiments, the extractant can be determined based on the boiling point (above the boiling point of the unsaturated acid) and their basicity and/or Hansen parameters.

In order to realize cost-effective separation (recovery) of the extractive solvent from the unsaturated carboxylic acid by e.g. distillation, advantageously the extractive solvent has a boiling point at atmospheric pressure that is at least 5° C. higher, preferably at least 10° C. higher, more preferably at least 20° C. higher than the boiling point of the unsaturated carboxylic acid. In all embodiments, the extraction solvent has boiling point greater than the compounds to be separated.

Thus, for the recovery of acrylic acid, which has a boiling point of 141° C. at atmospheric pressure, it is preferred that the extractive solvent has a boiling point of at least 146° C. Preferably, it has a boiling point of at least 150° C., more preferably at least 160° C., even more preferably at least 170° C.

From an economic perspective, it is preferred that the extractive solvent has a boiling point that does not exceed 300° C., more preferably does not exceed 280° C., even more preferably does not exceed 250° C., most preferably does not exceed 225° C., at atmospheric pressure, in order to avoid excessive heating expenditure.

In some embodiments, the basicity may be determined by the pKa of the protonated form of the solvent: the higher the pKa, the lower the acidity of the protonated solvent and the higher the basicity of the unprotonated solvent. In some embodiments, the extractive solvent may have a protonated form with pKa above (−5), preferably above (−2), more preferably above 0 and most preferably above 2.

In other embodiments, an alternative measure for basicity is the proton affinity. The extractive solvent may have a proton affinity above 700 kJ/mol, preferably above 800, more preferably above 850, and most preferably 900 kJ/mol.

In other embodiments, the extractive solvent may be selected based on its polarity parameters and, more specifically, based on distances in Hansen parameter space to the saturated and unsaturated carboxylic acids. Indeed, the saturated and unsaturated carboxylic acids are separated from one another by a distance of 6.6 [Mpa^{1/2}] in the Hansen parameter space.

Hansen solubility parameters (HSP) can be used as a means for predicting the likeliness of one compound (solvent) dissolving in another. More specifically, each compound is characterized by three Hansen parameters, each generally expressed in MPa^{0.5}: δ_{d}, denoting the energy from dispersion forces between molecules; δₚ, denoting the energy from dipolar intermolecular forces between molecules; and δₕ, denoting the energy from hydrogen bonds between molecules. The affinity between compounds can be described using a multidimensional vector that quantifies these solvent atomic and molecular interactions, as a Hansen solubility parameters (HSP) distance Rₐ which is defined in Equation (1):

(Rₐ)²=4(δ_{d2}−δ_{d1})²+(δₚ₂−δₚ₁)²+(δₕ₂−δₕ₁)² (1)

wherein
Rₐ=distance in HSP space between compound 1 and compound 2 (MPa^{0.5})
δ_{d1}, δₚ₁, δₕ₁=Hansen (or equivalent) parameter for compound 1 (in MPa^{0.5})
δ_{d2}, δₚ₂, δₕ₂=Hansen (or equivalent) parameter for compound 2 (in MPa^{0.5})

Thus, in the context of the present invention, a good extractive solvent will show a smaller value for Rₐ versus the unsaturated component (e.g. acrylic acid) than versus the saturated one (e.g. propionic acid).

Hansen solubility parameters for numerous solvents can be found in, among others, CRC Handbook of Solubility Parameters and Other Cohesion Parameters, Second Edition by Allan F. M. Barton, CRC press 1991; Hansen Solubility Parameters: A User's Handbook by Charles M. Hansen, CRC press 2007. It is also explained in these handbooks how analogous, equivalent solubility parameters have been derived by alternative methods to the original Hansen method, resulting in similarly useful parameters such as Hoy's cohesion parameters for liquids.

It is preferred that the absolute difference in Hansen solubility parameter distance Rₐ with respect to the unsaturated and saturated acid |ΔRa| as determined at 25° C. is 12 MPa^{1/2} or less, preferably 10 MPa^{1/2} or less, more preferably 8 MPa^{1/2} or less, most preferably 5 MPa^{1/2} or less.

Extractive solvents with a shorter distance vs. unsaturated than saturated carboxylic acids (ΔRa>0) should interact more with the former than the latter and thereby entrain the unsaturated acid as bottom stream.

The following ΔRa values are illustrative for the separation of acrylic acid from propionic acid. In some embodiments, the extractive solvent may be, but not limited to: phenolic components and aromatic alcohols, such as phenol (ΔRa=6.3), cresol (ΔRa=4.2), ethyl phenol and phenoxy ethanols (ΔRa=5-5.5) or salicylic acid or esters (ΔRa=5.5-6.1), benzyl alcohol (ΔRa=5.7) and phenyl ethanol (ΔRa=5.2); aromatic esters, such as methyl benzoate (ΔRa=1.1); sulfur oxides, such as sulfolane (ΔRa=3.5), phosphine oxide and nitrogen-oxides; amides, such as n-methyl pyroliddone (ΔRa=1.2) and n-methyl acetamide (ΔRa=0.6), lactam, amines, imines, pyridines and related N-components; diols, such ethylene glycol, propylene glycols and 1,4 butanediol (ΔRa=2.8-3.4); lactones, such as gamma-valerolactone and -butyrolactone (ΔRa=2.1); unsaturated aldehydes and ketones such as furfural (ΔRa=1.3).

In some embodiments, there may be a correspondence between basicity and polarity. While not wishing to be bound by theory, this correspondence may not be accidental because basic centres also confer significant polarity. However, the correspondence may be partial since acidic centers also confer polarity. Lack of polarity is mainly found in neutral molecules that show no significant acidity and basicity. In fact, basicity and polarity are likely to play a role in the selective interaction with unsaturated acids.

In some embodiments, the extractive solvent may include basic chemical functionalities, such as but not limited to: alcohols and ethers (incl. diols, polyols, hydroxyethers and polyethers); sulfoxide, including sulfolane, nitrogen-oxide and phosphine-oxide; amides and lactams; amines, imine, pyridine and other acyclic and cyclic N-components; and phosphine.

In other embodiments, the extractant is a polar solvent. The polar solvent is typically a polar organic solvent. Any suitable polar organic solvent may be employed. Thus the extraction solvent may comprise a compound selected from an alcohol, an aldehyde, a ketone, an ether, a carboxylic acid, an ester, a carbonate, an acid anhydride, an amide, an amine, a heterocyclic compound, an imine, an imide, a nitrile, a nitro compound, a sulfoxide, and a haloalkane, wherein the compound is a liquid under the conditions of the extraction. The extraction solvent may comprise two or more polar organic solvents. The extraction solvent may consist essentially of one or more polar organic solvents. The extraction may consist essentially of a single polar organic solvent. However, the extraction solvent may also be a binary solvent or a multinary solvent as discussed below.

In some embodiments, the extraction solvent may comprise any C₆₋₁₀ monoalcohol or any C₂₋₁₀ polyalcohol. The alcohol may be an alcohol of formula ROH or HOR′OH, wherein R and R′ are C6-10 and C2-10 groups selected from unsubstituted or substituted alkyl, unsubstituted or substituted alkenyl, unsubstituted or substituted alkynyl, unsubstituted or substituted cycloalkyl, and unsubstituted or substituted aryl. The R and R′ can be linear, branched, cyclic, saturated, unsaturated, and may include aromatics.

Non-limiting examples of alcohols which the extraction solvent may comprise include: monohydric alcohols such as, cyclohexanol, hexanol, heptanol and octanol; and polyhydric alcohols such as ethane-1,2-diol (ethylene glycol), propane-1,2-diol (propylene glycol), propane-1,3-diol, propane-1,2,3-triol (glycerol), butanediol, isobutanediol, tertbutanediol, butanetriol, pentanediol, methylbutanediol, hexanediol, hexanetriol. For compounds wherein the positions of hydroxy groups are not specified, alcohols having each of the positions are covered. Thus, butanediol includes butane-1,2-diol, butane-1,3-diol, butane-1,4-diol and butane-2,3-diol. Ethane-1,2-diol (ethylene glycol), propane-1,2-diol (propylene glycol), propane-1,3-diol, and butanediol are examples of dihydric alcohols. In particular, the alcohol which the extraction solvent comprises may be selected from cyclohexanol, hexanol, ethylene glycol, propylene glycol, and propane-1,3-diol. For instance, the extraction solvent may comprise a polar organic solvent selected from cyclohexanol, hexanol, ethylene glycol and propylene glycol.

In some embodiments, the extraction solvent may comprise any C7+ aldehyde. An aldehyde typically has the structure R—CHO. Lower carbon number aldehydes may be used if they contain another polar group (e.g. —OH). The R can be linear, branched or cyclic. The R may also be saturated or unsaturated, including aromatics.

In some embodiments, the extraction solvent may comprise any C6+ cyclic ketone or any C7+ acyclic ketone. Lower ketones may be used if they contain another polar group (e.g. —OH). A ketone typically has the structure R—C(O)—R′, with R and R′ being a hydrocarbon that is linear, branched or cyclic, saturated, or unsaturated, including aromatics. Moreover, R and R′ can be connected to form a cyclic ketone.

In some embodiments, the extraction solvent may comprise any C8+ ether or may have a lower carbon number if they contain another polar group (e.g. aromatic group in anisol). An ether typically has the structure R—O—R′, with R and R′ being a hydrocarbon that is linear, branched or cyclic, saturated, or unsaturated, including aromatics. Moreover, R and R′ can be connected to form a cyclic ether.

In some embodiments, the extraction solvent may comprise any C8+ ester. An ester typically has the structure R—COO—R′, with R and R′ being a hydrocarbon that is linear, branched or cyclic, saturated, or unsaturated, including aromatics. Moreover, R and R′ can be connected to form a cyclic ester.

In some embodiments, the extraction solvent may comprise any C6+ carbonate. A carbonate typically has the structure R—OC(O)OR′, with R and R′ being a hydrocarbon that is linear, branched or cyclic, saturated, or unsaturated, including aromatics. Moreover, R and R′ can be connected to form a cyclic carbonate. In other embodiments, the extraction solvent may be a C3+ cyclic carbonate, e.g. ethylene carbonate

In some embodiments, the extraction solvent may comprise any C5+ acid anhydride. An example of the acid anhydride which the extraction solvent may comprise is maleic anhydride.

In some embodiments, the extraction solvent may comprise any C1-10 amide. An amide typically has the structure R—C(O)—N(R′)₂, with R and R′ being a hydrocarbon that is linear, branched or cyclic, saturated, or unsaturated, including aromatics. Moreover, R and R′ can be connected to form a cyclic acid amide. Non-limiting examples of the amide which the extraction solvent may comprise include formamide, N-methyl formamide, dimethyl formamide, dimethyl acetamide, N-vinylacetamide, pyrrolidone, N-methyl pyrrolidone, and N-vinyl pyrrolidone.

In some embodiments, the extraction solvent may comprise any C7+ mono-amine and may have a lower carbon number for diamines and triamines. An amine typically has the structure RNH₂, RR′NH, and RR′R″N, with R, R′ and R″ being a hydrocarbon that is linear, branched or cyclic, saturated, or unsaturated, including aromatics. R, R′ and R″ can be connected to one another to form a cyclic amine. The amine may be a C2-10-alkylenediamine. Non-limiting examples of the amine which the extraction may comprise include diethyl,propyl-amine, ethyl,cyclopentyl amine, methyl-cyclohexyl amine tripropylamine, tributylamine, ethylenediamine, propylenediamine, diethylenetriamine, morpholine, piperidine, and quinoline.

In some embodiments, the extraction solvent may comprise a heterocyclic compound wherein the boiling point is greater than the compounds to be separated. The heterocyclic compound may be any compound comprising a ring, which ring comprises a heteroatom selected from N, P, O and S.

In some embodiments, the extraction solvent may comprise any C4-10 imine or any C4-10 imide wherein the boiling point is greater than the compounds to be separated.

In some embodiments, the extraction solvent may comprise any C5+ nitrile. In some embodiments, the extraction solvent may comprise any C5-10 nitro compound.

In some embodiments, the extraction solvent may comprise any C2-10 sulfoxide compound. For instance, the extraction solvent may comprise dimethylsulfoxide (DMSO). The extraction solvent may comprise diethylsulfoxide or methylethylsulfoxide as well as sulpholane.

In some embodiments, the extraction solvent may comprise any C2-10 haloalkane.

In some embodiments, the extractive solvent is N-methyl pyrrolidone, particularly for separating acrylic acid from propionic acid.

Any of the solvent compounds listed above (an alcohol, an aldehyde, a ketone, an ether, a carboxylic acid, an ester, a carbonate, an acid anhydride, an amide, an amine, a heterocyclic compound, an imine, an imide, a nitrile, a nitro compound, a sulfoxide, or a haloalkane) may be substituted or unsubstituted. Typically, the solvent compounds are unsubstituted.

Based on the criteria as provided herein the skilled person will be capable of selecting suitable extractive solvents from each of these classes of oxygen-containing organic compounds for the separation of same carbon number saturated carboxylic acids from unsaturated carboxylic acids.

In one embodiment, the recovery of saturated carboxylic acid from unsaturated carboxylic acid is described as providing a liquid or vaporous aqueous stream comprising saturated carboxylic acid and unsaturated carboxylic acid; contacting said aqueous stream comprising saturated carboxylic acid and unsaturated carboxylic acid with an extractive solvent in an extractive distillation unit, to produce a first stream comprising extractive solvent and saturated carboxylic acid and a second stream comprising unsaturated carboxylic acid; feeding said first stream comprising extractive solvent and saturated carboxylic acid to a solvent recovery unit, to produce a third stream comprising saturated carboxylic acid and a fourth stream comprising extractive solvent; recycling at least a portion of the fourth stream comprising extractive solvent to the extractive distillation unit, wherein the extractive solvent is wherein the extractive solvent is selected from a C2-10 amide, a C2-10 sulfoxide, or mixtures.

It is possible to combine the extractive solvent with one or more other solvents. In some embodiments, the other solvent should have a higher boiling point than the unsaturated carboxylic acid to form a miscible mixture with the extractant and the unsaturated carboxylic acid.

In one embodiment, a mixture of two or more extractive solvents as defined herein are used. In another embodiment, an extractive solvent as defined herein is combined with one or more solvents selected from carboxylic esters, ethers, aldehydes, or ketones. When one or more extractive solvents as defined herein are used in admixture with another solvent not according to the invention, it is preferred that the one or more extractive solvents as defined herein are present in a concentration of at least 40 wt %, more preferably at least 50 wt %, even more preferably at least 70 wt %, most preferably at least 80 wt % or 90 wt % based on total weight of the solvent mixture. In one embodiment, the one or more extractive solvents as defined herein are used in the absence of amine compounds. In one embodiment, the extractive solvent is employed in the absence of any other solvent not according to the invention. In order to avoid loss of solvent with acrylic acid, it is further preferred that if a mixture of solvents is used, that such mixture contains less than 20 wt %, more preferably less than 10 wt %, even more preferably less than 5 wt %, most preferably less than 2 wt %, based on total weight of the solvent mixture, of a solvent having a boiling that is less than 5° C. higher than the boiling point of the unsaturated acid.

Depending on, among others, the concentration of unsaturated carboxylic acids in the aqueous feed stream, the amount of extractive solvent employed in the extractive distillation process may vary within wide ranges, for example in a ratio (wt/wt) of extractive solvent to saturated carboxylic acid supplied to the extractive distillation unit in the range of from 100:1 to 0.1:1, preferably in the range of from 50:1 to 0.25:1, more preferably in the range of from 40:1 to 0.5:1, most preferably in the range of from 10:1 to 1:1.

Advantageously, substantially all of the unsaturated carboxylic acid present in the vaporous or liquid aqueous feed stream of the extractive distillation unit exits said extractive distillation unit in the extractive solvent stream. Typically, at least 90 wt %, preferably at least 95 wt %, more preferably at least 99 wt %, even more preferably at least 99.5 wt %, yet even more preferably at least 99.8 wt %, most preferably at least 99.9 wt % of unsaturated acid present in the feed stream of the extractive distillation unit is recovered in the extractive solvent stream of said extractive distillation unit. Furthermore, it is preferred that the extractive solvent entrains substantially none of the saturated acid (or ester) present in the gaseous or liquid aqueous feed stream of the extractive distillation unit.

Preferably, the extractive solvent effluent stream of the extractive distillation unit comprises saturated and unsaturated carboxylic acid (or ester) in a ratio of less than 1:1, more preferably less than 0.5:1, even more preferably less than 0.1:1, yet even more preferably less than 0.05:1, most preferably less than 0.01:1.

In the solvent recovery unit, unsaturated carboxylic acid is removed from the extractive solvent resulting in a product stream comprising unsaturated carboxylic acid and another stream comprising the extractive solvent now depleted of unsaturated carboxylic acid.

In the solvent recovery unit, recovery of the extractive solvent, and of optional other solvents present, is typically effectuated by distilling the effluent stream of the extractive distillation unit comprising unsaturated carboxylic acid and extractive solvent, resulting in a top stream comprising unsaturated carboxylic acid and a bottom stream comprising the extractive solvent. Distillation may be carried out in any distillation unit known to the skilled that is suitable for separating extractive solvent from unsaturated carboxylic acid, and it is within the ability of one skilled in the art to select appropriate operating conditions for obtaining a desired degree of product purity and/or solvent recovery.

Typically, the temperature in the solvent recovery unit would vary depending on the boiling point of the unsaturated acid and extractive solvent.

In one embodiment, the top temperature in the solvent recovery unit is at least 0° C., preferably at least 10° C., more preferably at least 20° C., most preferably at least 30° C. above the condensation temperature of the unsaturated carboxylic acid at operating pressure. In one embodiment, the bottom temperature in the solvent recovery unit is at most 20° C., preferably at most 10° C., more preferably at most 5° C., most preferably at most 0° C. below the condensation temperature of the extractive solvent at operating pressure.

Typically, the pressure is at least 100%, more preferably at least 110%, even more preferably at least 120%, most preferably at least 130% of the condensation pressure of the extractive solvent at operating bottom temperature. Typically, the pressure is at most 100%, preferably at most 90%, more preferably at most 80%, even more preferably at most 70%, most preferably at most 50% of the condensation pressure of unsaturated acid at operating top temperature.

In one embodiment that uses an extractive solvent that contains an alcohol function, steam is fed at the bottom of the solvent regeneration unit to hydrolyze any esters that may have been formed by esterification of the unsaturated carboxylic acid with a component of the solvent mixture.

It is preferred that at least 80 wt %, more preferably at least 90 wt %, even more preferably at least 95 wt %, yet even more preferably at least 98 wt % of the saturated carboxylic acid present in the stream fed to the solvent recovery unit comprising unsaturated carboxylic acid and extractive solvent is recovered.

It is further preferred that at least 80 wt %, more preferably at least 90 wt %, even more preferably at least 95 wt %, yet even more preferably at least 98 wt % of the solvent present in the stream fed to the solvent recovery unit comprising unsaturated carboxylic acid and extractive solvent is recovered.

Typically, the unsaturated carboxylic acid product stream of the solvent recovery unit comprises unsaturated carboxylic acid in a concentration of at least 70 wt %, preferably at least 80 wt %, more preferably at least 90 wt %, more preferably at least 95 wt %, even more preferably at least 99 wt %, yet even more preferably at least 99.5 wt %, most preferably at least 99.9 wt %.

Based on the amount of unsaturated carboxylic acid present in the aqueous stream provided to the extractive distillation unit, at least 50 wt %, more preferably at least 75 wt %, even more preferably at least 90 wt %, yet even more preferably at least 95 wt %, most preferably at least 99 wt % of unsaturated carboxylic acid is recovered in the process as defined herein.

In a preferred embodiment, at least a portion of the stream of the solvent recovery unit comprising the extractive solvent, typically the bottom stream of a distillation unit, is recirculated to the extractive distillation unit. Typically, at least 20 wt %, preferably at least 50 wt %, more preferably at least 70 wt %, most preferably at least 90 wt % of the recovered solvent stream is recirculated to the extractive distillation unit. In one embodiment, the entire bottom stream comprising the extractive solvent is recirculated to the extractive distillation unit.

In the extractive distillation column, a top stream comprising or substantially consisting of saturated carboxylic acid, water and optionally other gases lighter than water, is typically produced. Saturated carboxylic acid may be recovered from this top stream using a condensation step, for example by cooling down the top stream of the extractive distillation unit to a lower temperature, for example room temperature, so that the unsaturated carboxylic acid can be recovered as a liquid stream.

The saturated carboxylic acid vapour top stream of the extractive distillation unit may further comprise entrained extractive solvent. Typically, said top stream of the extractive distillation unit comprises no more than 3 vol %, preferably at most 1 vol %, more preferably at most 0.3, even more preferably at most 0.1, most preferably at most 0.01 vol % of entrained extractive solvent.

The top stream comprising unsaturated carboxylic acid originating from the solvent recovery unit may be further treated downstream, for example to further remove water by liquid/liquid extraction, (azeotropic) distillation, pervaporation, etc., and/or other purification methods available in the art to obtain the purity and specifications for unsaturated carboxylic acid products according to market requirements.

The FIGURE depicts a process flow diagram for an embodiment. A stream 102 comprising saturated and unsaturated carboxylic acid is fed to an extractive distillation column 100 to which further an extractive solvent 104 is fed. Unsaturated carboxylic acid is extracted by the extractive solvent, which exits the extractive distillation column as “fat” solvent stream 108. A vapour stream comprising saturated carboxylic acid compounds exits the extractive distillation column as stream 106.

Stream 108 comprising extractive solvent and extracted unsaturated carboxylic acid is supplied to a solvent regeneration (recovery) unit, comprising a distillation unit 110. Unsaturated carboxylic acid leaves distillation unit 110 as top stream 112, while extractive solvent now depleted of saturated carboxylic acid exits distillation unit 110 as bottom stream 114. The unsaturated carboxylic acid-depleted extractive solvent stream 114 may be fully or partially recirculated to extractive distillation column 100. Unsaturated carboxylic acid stream 112 may be further purified downstream.

The invention is further illustrated by the following Example.

### EXAMPLE 1

### Vapour-Liquid Equilibrium Data Saturated/Unsaturated Carboxylic Acid Streams with Extraction Solvent Systems

Chemicals, from commercial sources as shown in Table 1, were dried over molecular sieve. The acids were used without any further purification. In order to avoid polymerization, acrylic acid was stabilized with 1000 wt ppm of phenothiazine. The properties of the pure components are listed in Table 1.

TABLE 1

| Component | supplier | purity/% GC |
|---|---|---|
| Acrylic acid | Sigma-Aldrich | 99 |
| Propionic acid | Fisher Chemicals | >98 |
| N-methyl-pyrrolidone (NMP) | Fluka | >98 |
| Lauric acid | Acros | 99 |

The isobaric VLE data were measured by means of a dynamic method using a Swietoslawski ebulliometer as described by Rogalski and Malanowski, Fluid Phase Equilib. 5 (1980) 97-112. At a given pressure, which is regulated by an electronic pressure control, the boiling temperature of a mixture can be measured. When phase equilibrium is reached, i.e. a stable circulation is achieved, and the boiling temperature is constant, the concentrations of both phases can be determined by taking samples from the liquid and the condensed vapor phase and gas chromatographic analysis.

For the binary system of acrylic acid (unsaturated carboxylic acid)+propionic acid (saturated carboxylic acid), VLE data were measured at constant pressures of 100 and 250 mbar for different feed compositions. The results are listed in Tables 2 and 3, where xi represents the concentration of i in the liquid phase, yi represents the concentration of I in the gas phase and Ki represents the distribution of i over both phases as xi/yi molar ratio. These tables confirm the difficulty of separating the two acids by simple distillation as both components distribute equally over the gas and liquid phase, i.e. K1 and K2 are close to 1.0, and consequently, the relative volatility α=K1/K2 ratio is close to 1.0.

TABLE 2
Experimental VLE (isobaric Txy) data for the binary
system acrylic acid (1) + propionic acid (2) at 100 mbar

| P/ | T/ | x₁/ | y₁/ | K₁ = | K₂ = | α = |
|---|---|---|---|---|---|---|
| bar | ° C. | (mol/mol) | (mol/mol) | y₁/x₁ | y₂/x₂ | K1/K2 |
| 0.1005 | 78.14 | 0.0000 | 0.0000 | | 1.000 | |
| 0.0998 | 79.72 | 0.0894 | 0.0900 | 1.007 | 0.999 | 1.008 |
| 0.0998 | 79.70 | 0.1766 | 0.1772 | 1.003 | 0.999 | 1.004 |
| 0.1003 | 80.31 | 0.2669 | 0.2638 | 0.988 | 1.004 | 0.984 |
| 0.1008 | 79.91 | 0.3978 | 0.4010 | 1.008 | 0.995 | 1.013 |
| 0.1000 | 80.63 | 0.4515 | 0.4444 | 0.984 | 1.013 | 0.971 |
| 0.1005 | 80.06 | 0.5683 | 0.5671 | 0.998 | 1.003 | 0.995 |
| 0.0998 | 80.17 | 0.6340 | 0.6265 | 0.988 | 1.020 | 0.969 |
| 0.1010 | 79.27 | 0.7559 | 0.7549 | 0.999 | 1.004 | 0.995 |
| 0.0998 | 78.98 | 0.8395 | 0.8414 | 1.002 | 0.988 | 1.014 |
| 0.1002 | 78.65 | 0.9234 | 0.9243 | 1.001 | 0.989 | 1.012 |
| 0.1000 | 78.75 | 1.0000 | 1.0000 | 1.000 | | |

TABLE 3
Experimental VLE (isobaric Txy) data for the binary
system acrylic acid (1) + propionic acid (2) at 250 mbar

| P/ | T/ | x₁/ | y₁/ | K₁ = | K₂ = | α = |
|---|---|---|---|---|---|---|
| bar | ° C. | (mol/mol) | (mol/mol) | y₁/x₁ | y₂/x₂ | K1/K2 |
| 0.2507 | 99.35 | 0.0000 | 0.0000 | | 1.000 | |
| 0.2508 | 101.42 | 0.0863 | 0.0869 | 1.008 | 0.999 | 1.009 |
| 0.2492 | 101.53 | 0.1758 | 0.1756 | 0.999 | 1.000 | 0.999 |
| 0.2499 | 101.97 | 0.2681 | 0.2633 | 0.982 | 1.007 | 0.975 |
| 0.2505 | 100.88 | 0.3962 | 0.3959 | 0.999 | 1.000 | 0.999 |
| 0.2506 | 102.36 | 0.4478 | 0.4459 | 0.996 | 1.003 | 0.993 |
| 0.2510 | 101.88 | 0.5685 | 0.5671 | 0.998 | 1.003 | 0.995 |
| 0.2522 | 102.41 | 0.6351 | 0.6230 | 0.981 | 1.033 | 0.950 |
| 0.2526 | 101.09 | 0.7579 | 0.7558 | 0.997 | 1.009 | 0.988 |
| 0.2500 | 101.14 | 0.8400 | 0.8399 | 1.000 | 1.000 | 1.000 |
| 0.2506 | 100.95 | 0.9243 | 0.9235 | 0.999 | 1.011 | 0.988 |
| 0.2500 | 100.82 | 1.0000 | 1.0000 | 1.000 | | |

Ternary data were measured for the same binary compositions mixed with 50 wt. % of NMP or lauric acid. During the measurements with NMP at 100 mbar (boiling points above 110° C.), polymerization was observed by a viscous liquid mixture inside the ebulliometer. Thus, the pressure had to be reduced to 25 mbar for these experiments. The results for both ternary systems are listed in Tables 4 and 5.

As can be seen in Tables 2 and 3, the relative volatility of acrylic acid vs. propionic acid (α=K1/K2) for the binary system are too close to 1 to separate acrylic acid vs. propionic acid efficiently. By adding NMP (Table 4), the relative volatility of acrylic acid is lowered to about 0.7, which allows a top product of propionic acid to be distilled from acrylic acid/NMP mixture, making the separation feasible. However, this is not the case when lauric acid is used (Table 5), thus there is an advantage to using NMP.

TABLE 4
Experimental VLE (isobaric Txy) data for the ternary system
acrylic acid (1) + propionic acid (2) + NMP (3) at 25 mbar

| | | x₁/ | x₂ | y₁/ | y₂/ | | | | α = |
|---|---|---|---|---|---|---|---|---|---|
| P/ | T/ | (mol/ | (mol/ | (mol/ | (mol/ | K₁ = | K₂ = | K₃ = | K1/ |
| bar | ° C. | mol) | mol) | mol) | mol) | y₁/x₁ | y₂/x₂ | y₃/x₃ | K2 |
| 0.0250 | 79.42 | 0.0000 | 0.5569 | 0.0000 | 0.9077 | | 1.630 | 0.2084 | |
| 0.0256 | 80.34 | 0.0598 | 0.5066 | 0.0655 | 0.8430 | 1.096 | 1.664 | 0.2108 | 0.659 |
| 0.0253 | 79.33 | 0.1088 | 0.4612 | 0.1334 | 0.7815 | 1.227 | 1.694 | 0.1978 | 0.724 |
| 0.0252 | 81.27 | 0.1701 | 0.3986 | 0.2105 | 0.7194 | 1.237 | 1.805 | 0.1625 | 0.685 |
| 0.0251 | 80.28 | 0.2290 | 0.3454 | 0.2918 | 0.6391 | 1.274 | 1.850 | 0.1624 | 0.689 |
| 0.0253 | 79.68 | 0.2855 | 0.2930 | 0.3696 | 0.5641 | 1.295 | 1.925 | 0.1573 | 0.673 |
| 0.0253 | 80.08 | 0.3434 | 0.2474 | 0.4518 | 0.4659 | 1.316 | 1.883 | 0.2011 | 0.699 |
| 0.0251 | 82.69 | 0.3770 | 0.1979 | 0.5287 | 0.4039 | 1.403 | 2.041 | 0.1585 | 0.687 |
| 0.0257 | 81.98 | 0.4535 | 0.1434 | 0.6477 | 0.2989 | 1.428 | 2.084 | 0.1324 | 0.685 |
| 0.0250 | 81.37 | 0.4842 | 0.0986 | 0.7256 | 0.2161 | 1.499 | 2.192 | 0.1397 | 0.684 |
| 0.0253 | 83.79 | 0.5565 | 0.0456 | 0.8416 | 0.1057 | 1.512 | 2.318 | 0.1326 | 0.652 |
| 0.0263 | 84.42 | 0.5736 | 0.0000 | 0.9507 | 0.0000 | 1.657 | | 0.1155 | |

TABLE 5
Experimental VLE (isobaric Txy) data for the ternary system
acrylic acid (1) + propionic acid (2) + lauric acid (3) at 100 mbar

| | | x₁/ | x₂ | y₁/ | y₂/ | | | | α = |
|---|---|---|---|---|---|---|---|---|---|
| P / | T/ | (mol/ | (mol/ | (mol/ | (mol/ | K₁ = | K₂ = | K₃ = | K1/ |
| bar | ° C. | mol) | mol) | mol) | mol) | y₁/x₁ | y₂/x₂ | y₃/x₃ | K2 |
| 0.0994 | 87.54 | 0.0000 | 0.7283 | 0.00000 | 0.99741 | | 1.369 | 0.0095 | |
| 0.0995 | 88.39 | 0.0702 | 0.6574 | 0.09436 | 0.90427 | 1.344 | 1.376 | 0.0050 | 0.977 |
| 0.0996 | 88.01 | 0.1399 | 0.5881 | 0.19062 | 0.80695 | 1.363 | 1.372 | 0.0089 | 0.993 |
| 0.1005 | 85.01 | 0.2029 | 0.5265 | 0.27869 | 0.71909 | 1.374 | 1.366 | 0.0082 | 1.006 |
| 0.1001 | 86.13 | 0.2765 | 0.4509 | 0.38340 | 0.61541 | 1.387 | 1.365 | 0.0044 | 1.016 |
| 0.0998 | 88.51 | 0.3398 | 0.3833 | 0.46329 | 0.53409 | 1.363 | 1.393 | 0.0095 | 0.978 |
| 0.1001 | 88.27 | 0.4058 | 0.3183 | 0.55851 | 0.44036 | 1.376 | 1.383 | 0.0041 | 0.995 |
| 0.0999 | 86.49 | 0.4816 | 0.2494 | 0.65457 | 0.34466 | 1.359 | 1.382 | 0.0029 | 0.983 |
| 0.0999 | 88.79 | 0.5446 | 0.1863 | 0.74222 | 0.25706 | 1.363 | 1.380 | 0.0027 | 0.988 |
| 0.0997 | 89.52 | 0.6080 | 0.1237 | 0.82465 | 0.17351 | 1.356 | 1.402 | 0.0069 | 0.967 |
| 0.1001 | 89.11 | 0.6711 | 0.0605 | 0.91397 | 0.08402 | 1.362 | 1.389 | 0.0075 | 0.981 |
| 0.0997 | 89.14 | 0.7357 | 0.0000 | 0.99896 | 0.00000 | 1.358 | | 0.0040 | |

Other and further embodiments utilizing one or more aspects of the inventions described above can be devised without departing from the spirit of Applicant's invention. The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

While the invention is susceptible to various modifications and alternative forms, specific embodiments thereof are shown by way of examples herein described in detail. It should be understood, that the detailed description thereto is not intended to limit the invention to the particular form disclosed, but on the contrary, the intention is to cover all modifications, equivalents and alternatives falling within the spirit and scope of the present invention as defined by the appended claims.

We claim:

1. A process for separating saturated and unsaturated carboxylic acids, comprising providing a stream comprising same carbon number saturated and unsaturated carboxylic acids; contacting said stream with an extractive solvent in an extractive distillation unit, to produce a first stream comprising extractive solvent and unsaturated carboxylic acids and a second stream comprising saturated carboxylic acids, and feeding said first stream to a solvent recovery unit, to produce a third stream comprising unsaturated carboxylic acids and a fourth stream comprising extractive solvent, wherein the extractive solvent has a boiling point at atmospheric pressure that is at least 5° C. higher than the boiling point of the unsaturated carboxylic acid.

2. The process according to claim 1, further comprising recycling at least a portion of the fourth stream to the extractive distillation unit.

3. The process according to claim 1, wherein the extractive solvent has a pKa greater than (−5) or a proton affinity higher than 700 kJ/mol.

4. The process according to claim 1, wherein the extractive solvent has an absolute difference in Hansen solubility parameter distance Rₐ with respect to the unsaturated acid and saturated acid |ΔRa| as determined at 25° C. is less than 12 MPa^{1/2}.

5. The process according to claim 1, wherein the extractive solvent comprises a solvent selected from the group consisting of alcohols, ethers, esters, aldehydes, ketones, amides, amines, nitriles and sulfoxides.

6. The process according to claim 1, wherein the extractive solvent is a compound selected from the group consisting of NMP, dimethylsulfoxide, sulfolane, N-formyl-morpholine, N-alkyl-pyrrolidones.

7. The process according to claim 1, wherein the saturated and unsaturated carboxylic acids comprise fatty acids.

8. The process according to claim 1, wherein the saturated and unsaturated carboxylic acids comprise esters derived from carboxylic acids.

9. The process according to claim 1, wherein before contacting the liquid or vaporous stream with an extractive solvent in the extractive distillation unit, said stream is concentrated using reverse osmosis, carboxylic acid-selective pervaporation, adsorption-desorption using a solid adsorbent or liquid-liquid extraction (LLE), preferably liquid-liquid extraction (LLE).

10. The process according to claim 1, wherein at least 50 wt % of unsaturated carboxylic acid is recovered, based on the amount of saturated carboxylic acid present in the liquid or vaporous stream provided to the extractive distillation step.

11. The process according to claim 1, wherein at least 75 wt % of unsaturated carboxylic acid is recovered, based on the amount of saturated carboxylic acid present in the liquid or vaporous stream provided to the extractive distillation step.

12. The process according to claim 1, wherein at least 90 wt % of unsaturated carboxylic acid is recovered, based on the amount of saturated carboxylic acid present in the liquid or vaporous stream provided to the extractive distillation step.

13. The process according to claim 1, wherein at least 95 wt % of unsaturated carboxylic acid is recovered, based on the amount of saturated carboxylic acid present in the liquid or vaporous stream provided to the extractive distillation step.

14. The process according to claim 1, wherein at least 99 wt % of unsaturated carboxylic acid is recovered, based on the amount of saturated carboxylic acid present in the liquid or vaporous stream provided to the extractive distillation step.
